(19) United States
(12) Patent Application Publication
(10) Pub. No.: US 2023/0355381 A1
(43) Pub. Date: Nov. 9, 2023

(54) Detachable Cell Configuration for Valve in Valve

(71) Applicant: St. Jude Medical, Cardiology Division, Inc., St. Paul, MN (US)

(72) Inventors: William H. Peckels, Robbinsdale, MN (US); Heath Marnach, Minneapolis, MN (US)

(73) Assignee: St. Jude Medical, Cardiology Division, Inc., St. Paul, MN (US)

(21) Appl. No.: 18/297,711

(22) Filed: Apr. 10, 2023

Related U.S. Application Data
(60) Provisional application No. 63/339,034, filed on May 6, 2022.

(51) Int. Cl. A61F 2/24

(52) U.S. Cl. CPC A61F 2/2418; A61F 2/2433; A61F 2250/001; A61F 2250/0063; A61F 2220/0033; A61F 2250/0036; A61F 2210/0014; A61F 2230/0017

(57) ABSTRACT

A prosthetic heart valve includes a stent body and prosthetic leaflets. The stent body extends from an inflow end to an outflow end and includes an annulus section defining a first row of cells extending in a circumferential direction, the stent body being expandable from a delivery condition having a first diameter to a deployed condition having a second diameter larger than the first diameter. The prosthetic leaflets are mounted to the stent body allow flow in an antegrade direction but substantially block flow in a retrograde direction. The first row of cells is circumferentially continuous in the delivery condition and in the deployed condition, and the stent body is further expandable from the deployed condition to an open condition in which the first row of cells is circumferentially discontinuous.

### CROSS-REFERENCE TO RELATED APPLICATIONS

This application claims priority to U.S. Provisional Patent Application No. 63/339,034, filed May 6, 2022, the disclosure of which is hereby incorporated by reference herein.

### BACKGROUND OF THE DISCLOSURE

The present disclosure related to heart valve replacement. More particularly, the present disclosure relates to prosthetic heart valves having a possible discontinuous configuration to allow for insertion of new valve for a follow-on valve-in-valve procedure.

Prosthetic heart valves that are collapsible to a relatively small circumferential size can be delivered into the patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery. Prosthetic surgical heart valves, on the other hand, are typically sutured directly into a patient's native heart valve annulus in an open-chest, open-heart surgery in which the patient is placed on cardiopulmonary bypass.

When a collapsed prosthetic valve has reached its desired implant site in the patient (e.g., at or near the annulus of the patient's heart valve that is to be replaced by the prosthetic valve), the prosthetic valve can be deployed or released from the delivery apparatus and re-expanded to full operating size. For balloon-expandable valves, this generally involves releasing the valve, assuring its proper location, and then expanding a balloon positioned within the valve stent. For self-expanding valves, on the other hand, the stent automatically expands as the sheath covering the valve is withdrawn.

Early studies suggest that prosthetic heart valves can last ten to fifteen years after implantation. For a young person with a prosthetic heart valve replacement, there may be a need for additional surgery or another heart valve replacement later in life. The prosthetic heart valve may become damaged or worn out such that it ceases to function properly. If the implanted prosthetic heart valve fails to function properly, a new replacement prosthetic heart valve may be implanted in an attempt to resume normal functions. However, at the point at which the original implanted prosthetic heart valve needs replacement, patients are often too old and frail for invasive surgical procedure thus, a less traumatic valve-in-valve procedure (hereinafter referred to as “VIV procedure”) may be performed. In a VIV procedure, a new prosthetic heart valve is implanted inside of the prior-implanted prosthetic heart valve using a minimally invasive transcatheter procedure.

One challenge that arises from VIV procedures is that the existing structure of the prior-implanted prosthetic heart valve may limit the size of the transcatheter heart valve that can be implanted inside of the prior-implanted prosthetic heart valve. Thus, the size of the second implanted transcatheter heart valve may be too small to the meet the patient's desired or optimum blood flow requirements. This results in the phenomenon of patient-prosthesis mismatch (hereinafter referred to as “PPM”). PPM has been shown to be associated with increased mortality after VIV procedures. Thus, there exists a need for a mechanism by which implanted prosthetic heart valves can be expanded in vivo after so that the existing implant can accept a sufficiently sized VIV transcatheter valve and minimize the potential for PPM.

### BRIEF SUMMARY OF THE DISCLOSURE

According to one aspect of the disclosure, a prosthetic heart valve includes a stent body and a plurality of prosthetic leaflets. The stent body may extend from an inflow end to an outflow end in a longitudinal direction. The stent body may include a generally tubular annulus section defining a first row of cells extending in a circumferential direction, and the stent body may be expandable from a delivery condition having a first diameter to a deployed condition having a second diameter larger than the first diameter. The plurality of prosthetic leaflets may be mounted to the stent body and may be operative to allow flow in an antegrade direction from the inflow end to the outflow end, but to substantially block flow in a retrograde direction from the outflow end to the inflow end. The first row of cells may be circumferentially continuous in the delivery condition and in the deployed condition, and the stent body may be further expandable from the deployed condition to an open condition in which the first row of cells is circumferentially discontinuous.

According to another aspect of the disclosure, a method of implanting a first prosthetic heart valve includes loading the first prosthetic heart valve into a sheath of a delivery device so that a stent body of the first prosthetic heart valve is maintained in a delivery condition with a first diameter. The method may include advancing the first prosthetic heart valve through a patient until the first prosthetic heart valve is positioned adjacent a native heart valve of the patient. The method may further include deploying the first prosthetic heart valve from the sheath of the delivery device into the native heart valve of the patient so that the stent body of the first prosthetic heart valve expands to a deployed condition with a second diameter larger than the first diameter, the stent body being further expandable, upon the application of a predetermined force to an inner surface of the stent body, from the deployed condition to an open condition in which a first row of cells of the stent body is circumferentially discontinuous. The method may also include withdrawing the delivery device from the patient and completing the implantation of the first prosthetic heart valve without further expanding the stent body from the deployed condition to the open condition.

### BRIEF DESCRIPTION OF THE DRAWINGS

FIG. 1 is a side elevational view of a conventional prosthetic heart valve.

FIG. 2 is a perspective view of a stent for use as part of a prosthetic atrioventricular heart valve.

FIG. 2A is a side elevational view of an alternate version of the stent of FIG. 2 in a deployed condition, according to one embodiment of the disclosure.

FIG. 2B is an elevational side view of the detachable cell prosthetic heart valve of FIG. 2A in open condition, according to one embodiment of the disclosure.

FIG. 2C is a side view of a prosthetic atrioventricular valve that includes the stent of FIGS. 2A-B.

FIG. 3A is a highly schematic illustration of a section of connectors in delivery or deployed condition, according to one embodiment of the disclosure.

FIGS. 3B and 3C are highly schematic illustrations of sections of connectors while disconnecting into open condition, according to one embodiment of the disclosure.

### DETAILED DESCRIPTION

As used herein in connection with a prosthetic heart valve, the term “inflow end” refers to the end of the heart valve through which blood enters when the valve is functioning as intended, and the term “outflow end” refers to the end of the heart valve through which blood exits when the valve is functioning as intended. The term “circumferential,” when used in connection with a prosthetic heart valve, refers to the direction around the perimeter of the valve. Also, when used herein, the words “generally” and “substantially” are intended to mean that slight variations from absolute are included within the scope of the structure or process recited.

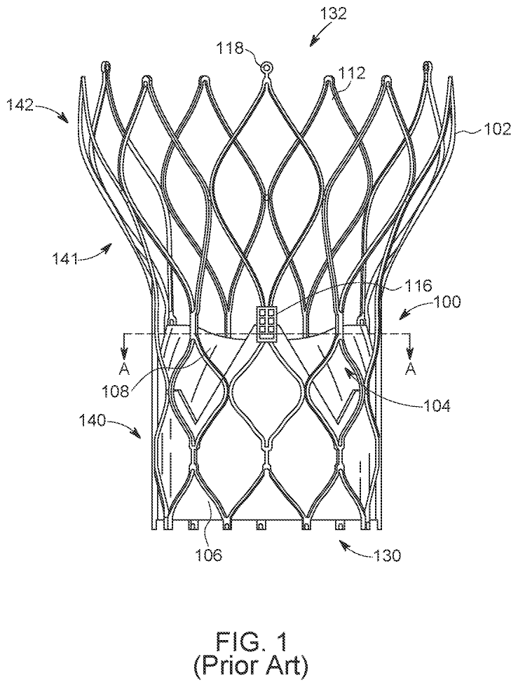

FIG. 1 shows a collapsible and expandable stent-supported prosthetic heart valve 100 known in the art. The prosthetic heart valve 100 is designed to replace the function of a native tricuspid, bicuspid or unicuspid valve of a patient, such as a native aortic valve. It should be noted that while the present disclosure is described predominantly in connection with prosthetic aortic valves and a stent having a shape as illustrated in FIG. 1, the concepts described herein may also be used with prosthetic bicuspid valves, such as prosthetic mitral valves, and with stents having different shapes, such as those having a flared or conical annulus section, a less-bulbous aortic section, a cylindrical shape (particularly for balloon-expandable valves) and the like, and a differently shaped transition section. Examples of collapsible prosthetic heart valves are described in International Patent Application Publication No. WO/2009/042196; U.S. Pat. Nos. 7,018,406; and 7,329,278, the disclosures of all of which are hereby incorporated herein by reference.

Prosthetic heart valve 100 will be described in more detail with reference to FIG. 1. Prosthetic heart valve 100 includes expandable stent 102, which may be formed from biocompatible materials that are capable of self-expansion, such as, for example, shape memory alloys such as nitinol. Stent 102 extends from proximal (also referred to as inflow) or annulus end 130 to distal (also referred to as outflow) or aortic end 132, and includes tubular annulus section 140 adjacent the proximal or inflow end and aortic section 142 adjacent the distal or outflow end. Annulus section 140 has a relatively small cross-section in the expanded condition, while aortic section 142 has a relatively large cross-section in the expanded condition. Preferably, annulus section 140 is in the form of a cylinder having a substantially round cross-section and a substantially constant diameter along its length. Transition section 141 may taper outwardly from annulus section 140 to aortic section 142. Each of the sections of stent 102 includes a plurality of cells 112 connected to one another in one or more annular rows around the stent. For example, as shown in FIG. 1, annulus section 140 may have two annular rows of cells 112 and aortic section 142 and transition section 141 may each have one or more annular rows of cells. Cells 112 in aortic section 142 may be larger than the cells in annulus section 140. The larger cells in aortic section 142 better enable prosthetic valve 100 to be positioned in the native valve annulus without the stent structure interfering with blood flow to the coronary arteries.

Stent 102 may include one or more retaining elements 118 at distal end 132 thereof, the retaining elements being sized and shaped to cooperate with retaining structures provided on the deployment device (not shown). The engagement of retaining elements 118 with retaining structures on the deployment device helps maintain prosthetic heart valve 100 in assembled relationship with the deployment device, minimizes longitudinal movement of the prosthetic heart valve relative to the deployment device during unsheathing or re-sheathing procedures, and helps prevent rotation of the prosthetic heart valve relative to the deployment device as the deployment device is advanced to the target location and the heart valve deployed. In some variations, retaining elements 118 may be disposed near proximal end 130 of heart valve 100.

Prosthetic heart valve 100 includes one or more prosthetic valve elements, such as valve assembly 104, preferably positioned in the annulus section 140 of stent 102 and secured to the stent. Valve assembly 104 includes cuff 106 and a plurality of leaflets 108, which collectively function as a one-way valve by co-apting with one another, generally allowing blood to flow in an antegrade direction while substantially blocking blood from flowing in a retrograde direction. As a prosthetic aortic valve, valve 100 has three leaflets 108. However, it will be appreciated that other prosthetic heart valves with which the active sealing mechanisms of the present disclosure may be used may have a greater or fewer number of leaflets.

Although cuff 106 is shown in FIG. 1 as being disposed on the luminal or inner surface of annulus section 140, it is contemplated that the cuff may be disposed on the abluminal or outer surface of the annulus section or may cover all or part of either or both of the luminal and abluminal surfaces. Both cuff 106 and leaflets 108 may be wholly or partly formed of any suitable biological material or polymer such as, for example, polytetrafluoroethylene (PTFE), ultra-high molecular weight polyethylene (UHMWPE), polyethylene terephthalate (PET), silicone, urethane, and combinations of the preceding materials.

Leaflets 108 may be attached along their belly portions to cells 112 of stent 102, with the commissure between adjacent leaflets attached to commissure attachment features (“CAFs”) 116. The particular size and shape of CAFs 116 may vary in different valves, for example valves with larger or smaller diameters may include CAFs that are sized or shaped different than the illustrated CAFs. As can be seen in FIG. 1, each CAF 116 may lie at the intersection of four cells 112 of stent 102, two of the cells being adjacent one another in the same annular row, and the other two cells being in different annular rows and lying in end-to-end relationship. Preferably, CAFs 116 are positioned entirely within the annulus section 140 of stent 102 or at the juncture of annulus section 140 and transition section 141. CAFs 116 may include one or more eyelets which facilitate the suturing of the leaflet commissure to the stent.

Prosthetic heart valve 100 may be used to replace, for example, a native aortic valve, a surgical heart valve, a repair device or a heart valve that has undergone a surgical procedure. The prosthetic heart valve may be delivered to the desired site (e.g., near the native aortic annulus) using any suitable delivery device. During delivery, the prosthetic heart valve is disposed inside the delivery device in the delivery condition. The delivery device may be introduced into a patient using a transfemoral, transapical, transseptal, transaortic, subclavian or any other percutaneous approach. Once the delivery device has reached the target site, the user may deploy prosthetic heart valve 100. Upon deployment, prosthetic heart valve 100 expands so that annulus section 140 is in secure engagement within the native aortic annulus.

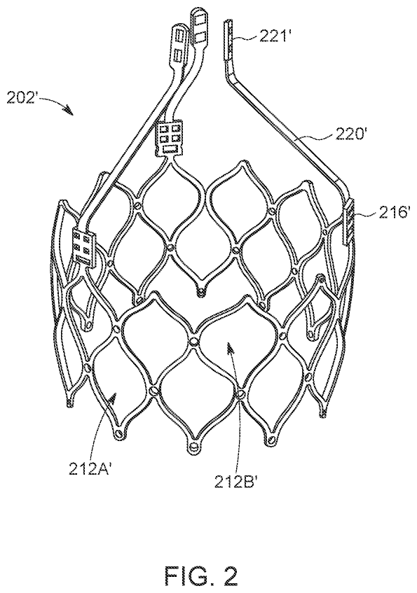

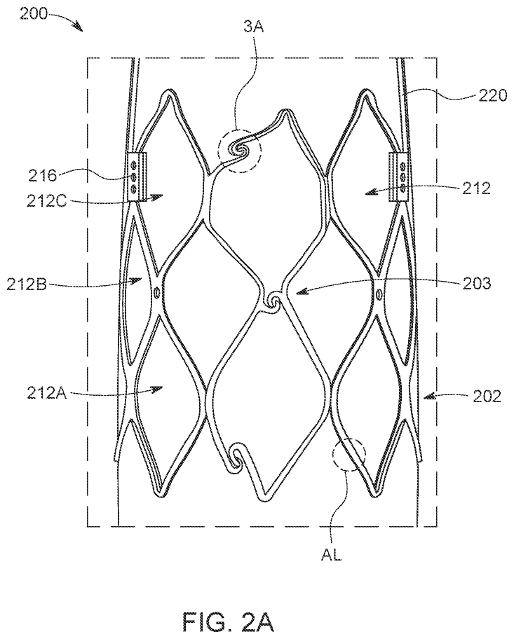

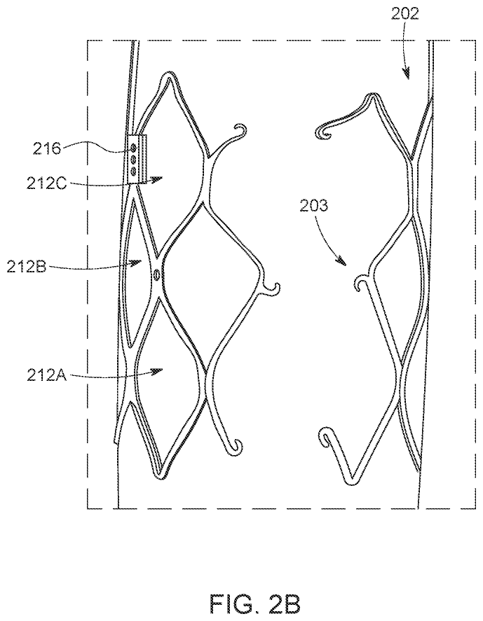

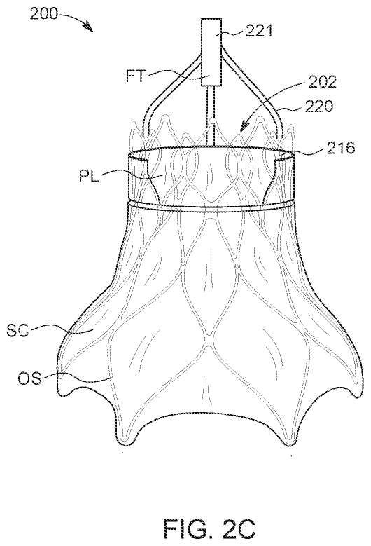

FIG. 2 is a perspective view of a stent 202′ that may be used as part of a prosthetic heart valve for replacement of a native atrioventricular valve, such as a mitral valve or tricuspid valve. Stent 202′ may be a self-expanding stent formed of a shape memory alloy such as nitinol, and may be formed by laser cutting the stent 202′ from a tube and shape-setting the stent 202′ to a desired shape. However, in some embodiments, stent 202′ may be formed as a plastically expandable (e.g. balloon expandable stent), including by forming the stent 202′ of a material such as stainless steel, cobalt chrome, or the like. In the illustrated embodiment, stent 202′ includes a plurality of struts forming two rows of generally diamond-shaped cells, including an inflow row of cells 212A′ and an adjacent outflow row of cells 212B′. If desired, apertures may be created in the struts at the position where adjacent cells within a row meet, as well as at the inflow and/or outflow apices of the cells. Such apertures may provide locations for securing a suture to the stent 202′ if desired. Stent 202′ may include a plurality (in this particular example, three) of commissure attachment features (“CAFs”) 216′ that may be used to fix ends of the prosthetic leaflets to the stent 202′. In the illustrated example, CAFs 216′ are generally rectangular with a plurality of eyelets formed therein to assist in passing one or more sutures through the CAFs 216′. The CAFs 216′ extend from outflow apices of three cells in the outflow row 212B′, preferably at equal intervals (e.g. 120 degrees apart from each other). From each CAF 216′ a tether 220′, in the form of an elongated strut, extends in the outflow direction with each tether 220′ angled radially inwardly so that the terminal free ends 221′ of the tethers 220′ converge to a point close to each other. The terminal free ends 221′ may each include one or more (two in the illustrated example) eyelets for assisting with suturing. For example, although not shown, a separate tether (e.g. formed of a braided synthetic fabric) may be positioned inside the converging terminal free ends 221′ and sutured to the terminal free ends 221′ to secure the metal tethers 220′ to the fabric tether. The fabric tether may be used to anchor the stent 220′ to an epicardial pad anchor such as that described in U.S. Pat. No. 10,610,354, the disclosure of which is hereby incorporated by reference herein. It should be understood that a prosthetic heart valve (e.g. a prosthetic mitral valve) incorporating stent 202′, similar to that shown and described in greater detail below in connection with FIG. 2C, may include an additional outer or anchoring stent, with the anchoring stent helping to prevent migration of the prosthetic heart valve into the left ventricle, and the fabric tether (and corresponding epicardial anchor) helping to prevent migration of the prosthetic heart valve into the left atrium.

FIG. 2A illustrates a portion of a stent 202 that is generally similar to stent 200 with two main exceptions, including an additional row of cells and a plurality of connectors described in greater detail below. Stent 200 may be used as part of a prosthetic heart valve 200, such as that shown in FIG. 2C, and stent 200 is illustrated in FIG. 2A in a deployed condition. In FIGS. 2A-B, stent 202 is shown with an opaque tube passing through a center thereof to provide a clearer picture of elements in the foreground. Further, FIGS. 2A-B omit from the view components that would otherwise be included in forming the prosthetic heart valve 200, including for example a skirt or cuff on the luminal and/or abluminal surface of the stent 202, and a plurality of prosthetic valve leaflets mounted within the stent 202. In the particular embodiment shown in FIGS. 2A-B, prosthetic heart valve 200 includes connectors 203 for holding the stent of the prosthetic heart valve 200 in a circumferentially continuous condition. The connectors 203 may be formed with a configuration so that the connectors 203 disconnect or “break away” from each other when subjected to predetermined force, while the struts forming the cells remain connected to each other when subjected to that predetermined force. The connectors 203 may take the general form of hooks, although other shapes and configurations may be suitable. In this example, the connectors 203 take the form of hooks.

In the depicted embodiment of FIG. 2A, prosthetic heart valve 200 includes an expandable stent 202 with a plurality of connectors 203. Stent 202 extends from an inflow end (toward the top of the view of FIG. 2A) to an outflow end (toward the bottom of the view of FIG. 2A). Stent 202 may be formed of a shape-memory material such as nitinol. However, in other embodiments, stent 202 may be a balloon-expandable stent and may be formed of materials capable of plastic deformation such as stainless steel, cobalt chromium, etc. The stent 202 may include a plurality of cells 212 connected to one another in one or more annular or circumferential rows around the stent 202, which may be similar in many aspects to stent 102 of FIG. 1. As noted above, in FIGS. 2A and 2B, there is a white background within the stent 202 to highlight the characteristics of the prosthetic heart valve 200. In practice, the prosthetic heart valve 200 would form a complete circumferential structure when the connectors 203 are engaged, generally similar to the stent 202′ of FIG. 2. In FIGS. 2A and 2B, the plurality of connectors 203 are arranged in a generally, although not exact, straight line along an axis parallel to the longitudinal axis of the stent 202. In other embodiments, the connectors 203 may be arranged exactly in a straight line along an axis parallel to the longitudinal axis of the stent 202. In other alternative embodiments, the plurality of connectors 203 may be arranged uniformly or non-uniformly spaced apart along the longitudinal direction in a skewed or zigzag pattern (not shown). In another alternative embodiment, there may be multiple columns of connectors 203 along the longitudinal axis of the stent 202 (not shown). In other words, while the illustrated embodiment shows a single connector 203 in each of the three rows of cells, in other embodiments, each row of cells may include more than one connector 203, including for example a second column of connectors positioned about 180 degrees apart from those illustrated in FIGS. 2A-B.

Still referring to FIG. 2A, stent 202 includes a first row of cells 212A at the inflow end of the stent, a second row of cells 212B adjacent the first row of cells 212A in an outflow direction of the stent 202, and a third row of cells 212C adjacent the second row of cells 212B in the outflow direction of the stent 202. However, more or fewer than three rows of cells may be provided (including two rows as shown in FIG. 2). Commissure attachment features 216 may be provided on the stent 202 to facilitate coupling prosthetic leaflets to the stent 202, generally similar to commissure attachment features 116 and 216′. In the illustrated embodiment, a total of three commissure attachment features 216 are provided in the third row of cells 212C, each commissure attachment feature 216 being positioned between a circumferentially adjacent pair of cells in the third row 212C. However, more or fewer commissure attachment features 216 may be provided depending on the number of prosthetic leaflets to be attached to the stent 202.

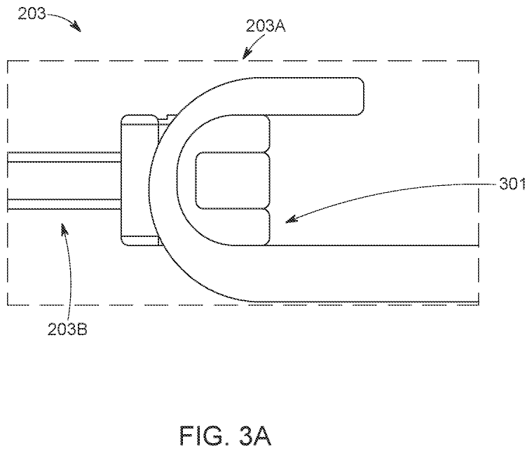

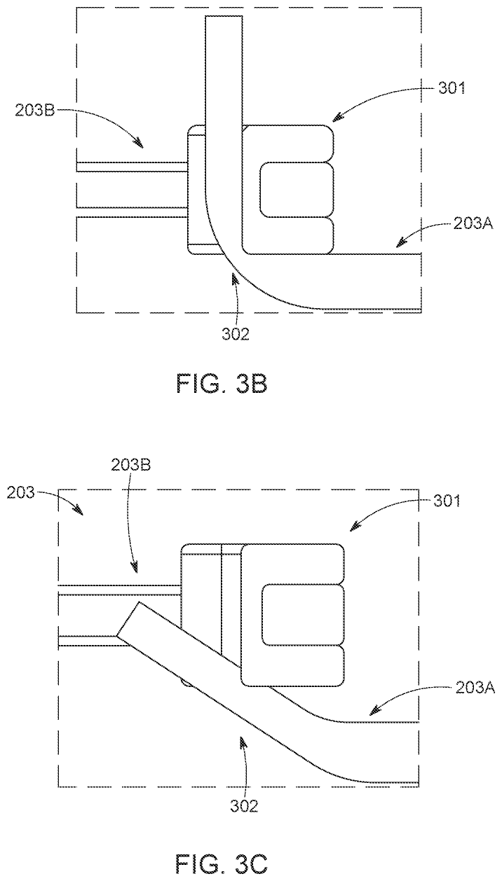

Still referring to FIG. 2A, at least one connector 203 is provided in association with each row of cells 212A-C. As is described in greater detail below, each connector 203 may form a part of a particular cell 212 so that, when the connectors 203 are in a connected or coupled condition, each associated row of cells 212A-C is in a circumferentially continuous condition. When the connectors 203 are in a disconnected or decoupled condition, each associated row of cells 212A-C may be in a circumferentially discontinuous condition, as shown in FIG. 2B. Referring now to FIG. 3A, each connector 203 may include a first connector portion 203A and a second connector portion 203B. The first connector portion 203A may be fixed to (or integral with) a first cell (or a portion thereof) and the second connector portion 203B may be fixed to (or integral with) a second cell (or a portion thereof). Further, in the embodiment shown in FIG. 2A, the cells are substantially diamond shaped with at least one connector 203 coupling cells at the circumferential point where the two cells meet, although other connector positions may be suitable. For example, FIG. 2A also shows connectors 203 positioned along a single strut forming one of the diamond-shaped cells. Still other positions for connectors 203 may be suitable. The term “circumferentially discontinuous” is used herein in the context of the “open” condition of stent 202 as shown in FIG. 2B. It should be understood that this term refers to a row of stent cells where the struts forming the cells in the row cannot be continuously traced circumferentially around the row of cells that forms the row. In other words, in the circumferentially continuous condition, a row of cells in the stent cannot be flattened on a table. However, in the circumferentially discontinuous condition, the row of cells may be able to be flattened on a table.

Before describing the connectors 203 in greater detail below, brief reference is made to FIG. 2C, which illustrates a prosthetic atrioventricular valve 200 (e.g. a prosthetic mitral valve) that incorporates stent 202. In addition to stent 202 (or stent 202′), prosthetic heart valve 200 includes a fabric tether FT within the terminal ends 221 of the metal tethers 220 (only a portion of the fabric tether FT shown in FIG. 2C). Three prosthetic leaflets PL are shown within the stent 202 and coupled to CAFs 216. Prosthetic heart valve 200 may also include a second or outer stent OS that flares outwardly toward the inflow end to help anchoring on the atrial side of the atrioventricular valve. Outer stent OS is preferably self-expanding but in some embodiments could be balloon expandable. The outer stent OS may include one or more rows of diamond-shaped cells, for example formed of nitinol, stainless steel, cobalt chrome, etc. The outer stent OS may be coupled to stent 202 via any suitable mechanism, for example including sutures that pass through apertures within the struts of both the outer stent OS and stent 202. Outer stent OS is also shown with a stent covering SC, which may be a tissue and/or fabric covering, and which may help provide a seal with the native valve annulus.

FIG. 3A-3C schematically illustrate different steps in the process of disengaging the connectors 203 to transition prosthetic heart valve 200 from a deployed or implanted condition to an open condition. In FIG. 3A, the prosthetic heart valve 200 is in a deployed or implanted condition and the first connector portion 203A and the second connector portion 203B are engaged. For example, each connector portion 203A and 203B may have a hook-shape that hook around one another to maintain the connector 203 in a coupled or engaged condition in the absence of applied forces. An isolator 301 may be positioned at the point or area of engagement between the first connector portion 203A and the second connector portion 203B. The isolator 301 may be made of tissue, fabric, or another nonabrasive material. As will be described below, during transition of the connectors 203 from the engaged or coupled condition to a disengaged or decoupled condition, the connector portions 203A, 203B may tend to drag or scrape against each other. It is generally not desirable to have two metal components drag or scrape against each other, particularly when in a patient's body, as this may cause metal to be released into the body and/or may weaken portions of the stent 202, leading to a potential for fracturing of components of the stent 202. However, with this configuration in which an isolator 301 is positioned between faces of the connector portions 203A, 203B that would directly contact each other in the absence of the isolator 301, the first connector portion 203A and second connector portion 203B remain capable of remaining engaged without being in direct contact with each other. And as the connector portions 203A, 203B transition to a decoupled or disconnected condition, metal-to-metal contact is avoided.

The connector 203 may be designed to allow the stent 202 to preferentially break in a controlled manner. In the illustrated embodiment, each connector portion 203A, 203B is formed integrally with the remainder of the structure of stent 202 (e.g. the stent 202 is formed by laser cutting a single tube of metal). In other embodiments, the connectors 203 may be non-integral with the stent 202 and coupled to the stent 202 separately after the stent 202 is otherwise formed. In the illustrated embodiment, each connector portion 203A, 203B has a first end coupled to (or formed integrally with) the body of the stent 202, and extends to a free end, with a middle section coupling the first end and the free end. The middle section may be shaped to “hook” back so that each connector portion 203A, 203B forms a general “J”-shape. In one embodiment, the first connector portion 203A has a thinned neck section 302 at the point of engagement of the connector 203. In other words, the first end of the first connector portion 203A may have a thickness, and the middle section may have a second thickness smaller than the first thickness. The second connector portion 203B may have a substantially uniform thickness. The thinned neck section 302 may help to facilitate uniform bending away of the first connector portion 203A from the second connector portion 203B upon application of a predetermined amount of force. In an alternative embodiment, the first connector portion 203A and second connector portion 203B may both have substantially uniform thicknesses. In yet another embodiment, both the first connector portion 203A and the second connector portion 203B include a thinned neck portion 302. In some embodiments, instead of forming the thinned neck portion 302 by decreasing the thickness of the tube that is laser cut in the areas of the thinned neck portion 302. In other embodiments, the strut width (e.g. circumferential direction of the tubing) may be reduced to form the thinned neck portion 302. In some embodiments, both the strut thickness and width may be decreased to form the thinned neck portion 302.

As discussed above, each of the connectors 203 may be sized and shaped to be substantially identical to one another such that each connector disconnects in substantially the same way upon the application of the same amount of radially outward force. Upon disconnecting and decoupling of the connectors 203, the circumference of the stent 202 becomes circumferentially discontinuous, such that the circumferential perimeter of the stent 202 cannot be linked with a continuous line.

Referring again to FIGS. 2A-B, stent 202 includes three rows of cells 212A-C, and a total of three connectors 203. In particular, one connector 203 is provided where two circumferentially adjacent cells in the second row 212B couple to each other. This may be referred to as a cell-to-cell connector 203. Another connector is provided about mid-way along one of the four struts that form the diamond shape of a cell 212 in each of the first row of cells 212A and third row of cells 212C. These two connectors may be referred to as strut-to-strut connectors. These three connectors are preferably nearly longitudinally aligned, although only the two strut-to-strut connectors 203 are directly longitudinally aligned, while the cell-to-cell connector 203 is slightly offset in the circumferential direction than the two strut-to-strut connectors. Preferably, the minimum number of connectors 203 necessary to allow for the relevant rows of cells of the stent 202 to be circumferentially discontinuous is provided. Referring back to FIG. 2A, the position of the connector 203 in the first row of cells 212A may be shifted one cell over to an alternate location AL shown in FIG. 2A. When placing the connector 203 at the alternate location AL, for example still as a strut-so-strut connector, a generally diagonal breakaway would occur (as each of the three connectors 203 would be positioned along a single diagonal line), as opposed to the more straight (or semi-straight) breakaway shown in FIG. 2B. Using such a diagonal breakaway configuration versus a straight or semi-straight breakaway configuration may provide different implant strength and/or ease of breakaway, depending on the particular configuration.

An exemplary use of prosthetic heart valve 200 is described below. Prosthetic heart valve 200 may be used to replace, for example, a native mitral or tricuspid valve, although it should be understood that other heart valves may instead be replaced using prosthetic heart valve 200, including an aortic or pulmonary valve replacement described in additional detail below. The prosthetic heart valve 200 may be delivered to the desired site (e.g., near the native mitral annulus) using any suitable delivery device, including a transapical delivery route. During delivery, the prosthetic heart valve 200 is disposed inside the delivery device in a collapsed or delivery condition. For example, the prosthetic heart valve 200 may be pulled through a funnel or otherwise crimped to a small diameter, with the prosthetic heart valve 200 being maintained in that small diameter by an overlying sheath of a delivery device. The prosthetic heart valve 200 while in the delivery condition is radially compressed to enable placement into the delivery device and through the vasculature and/or in a minimally invasive manner through the chest via transapical delivery. The prosthetic heart valve 200 has a relatively small profile in the delivery condition, for example small enough to be housed within a delivery device of about 18 French (6 mm), although this size is merely exemplary. The delivery device may be introduced into a patient using a transfemoral, transapical, transseptal, transaortic, subclavian or any other percutaneous approach. While in the collapsed or delivery condition, the connectors 203 remain in an engaged condition so that the rows of cells 212A-C of the stent 202 are circumferentially continuous. Once the delivery device has reached the target site, the user may deploy prosthetic heart valve 200, for example by retracting a delivery sheath to uncover the prosthetic heart valve 200, allowing the prosthetic heart valve 200 to expand into the native valve annulus. Upon deployment, prosthetic heart valve 200 expands into an expanded or deployed condition so that rows of cells 212A-C of the stent 202 of prosthetic heart valve 200 are in secure engagement within the native mitral annulus (if the mitral valve is the valve being replaced). As described above, the connectors 203 are engaged during the delivery and deployed conditions. In other words, at this point during the procedure, the function of prosthetic heart valve 200 remains generally similar to prosthetic heart valve 100 (albeit in this example prosthetic heart valve 200 is a mitral valve replacement), although the differences in structure allow for additional functionality described below.

It should be understood that the prosthetic heart valve 200 will encounter forces during the transition to the collapsed or delivery condition, during the delivery itself, during the transition from the collapsed or delivery condition to the expanded or deployed condition, and throughout the normal operating conditions of the prosthetic heart valve 200, including from beating of the heart, blood flow, pressure differentials across the prosthetic heart valve 200, etc. Despite the prosthetic heart valve 200 encountering these forces, the connectors 203 are designed to remain engaged until a threshold force is applied from within the stent 202. This threshold force is significantly greater than the typical forces experienced during implantation and normal operation of the valve. Thus, the connectors 203 are configured to decouple only upon an intentional application of the threshold force from the inside of the stent 202. When the threshold force is reached or exceeded, such as by expansion of a dilation balloon, the connectors 203 disconnect by “breaking away” from each other, enabling the rows of cells 212A-C of the stent 202 to decouple from one another. Although the term “break away” is used above, it should be understood that it is desirable that no stent material actually breaks away or otherwise becomes dislodged from the prosthetic heart valve 200 during application of the threshold force.

After a period of time of normal operation of the prosthetic heart valve 200, the prosthetic heart valve 200 itself may begin to deteriorate. For example, if prosthetic heart valve 200 is implanted into a young patient, the patient may have a post-implant life expectancy of 20, 30, 40 years or more. During this time, the prosthetic leaflets PL may become calcified or otherwise deteriorate in a way that causes regurgitation or other inefficiencies in the prosthetic heart valve 200. If prosthetic heart valve 200 deteriorates enough that the patient would benefit from a new prosthetic heart valve, particularly a minimally invasive transcatheter prosthetic heart valve, prosthetic heart valve 200 includes features to facilitate this secondary or VIV procedure. In such a transcatheter VIV procedure, the new prosthetic heart valve is advanced to the target site in a collapsed condition, typically using a transfemoral or transapical approach, and deployed within the failing prosthetic heart valve.

To perform the VIV procedure, essentially the same procedure may be used as with the procedure described above for the prosthetic heart valve 200 above (although any suitable delivery approach may be used for each individual procedure). For the VIV procedure, prior to implanting the new prosthetic heart valve, the user may utilize a transcatheter balloon device to apply at least the threshold force to the interior of the prosthetic heart valve 200 to transition the prosthetic heart valve 200 from the deployed condition to the open condition. For example, the balloon catheter may be advanced to the implanted prosthetic heart valve 200 using a transfemoral or transapical approach. The balloon catheter will be deflated as the user navigates it through the vasculature into the stent 202 of the prosthetic heart valve 200 to be replaced. When the balloon of the balloon catheter reaches the desired position within the stent 202 of the prosthetic heart valve 200, the user may inflate the balloon (e.g. by pushing saline into the balloon) to radially apply at least the threshold force to the stent 202. The balloon catheter may be specialized for this procedure. The radially outward force can be exerted on the stent 202 by a balloon or other expansion mechanism positioned inside the frame 200 either prior to or during a VIV procedure, although it preferably occurs just prior to the new prosthetic heart valve being implanted.

When the predetermined force is applied in the stent 202, the connectors 203 will break away and decouple, generally following the sequence shown in FIGS. 3A-C. As described above, the thinned neck portion of first connector portion 203A (if included) will predictably break away for second portion of connector 203B, thus creating a uniform break in the plurality of connectors 203 substantially simultaneously. As a result of the decoupling of the connectors 203, the rows of cells 212A-C of the stent 202 may circumferentially expand from a deployed position to a circumferentially discontinuous, open condition. The rows of cells 212A-C may be generally referred to as an annulus section, as this section of the stent 202 is generally for positioning within the native valve annulus.

If prosthetic heart valve 200 did not have the connectors 203 in the annulus portion of the stent 202 to allow for the annulus portion to “break” open into a circumferentially discontinuous condition, the second prosthetic heart valve might have less space available and thus may not be able to fully expand, or otherwise may need to be undersized to account for the additional structure of the original prosthetic heart valve 200. By “breaking” open, the secondary prosthetic heart valve will have relatively fewer space constraints. It should be understood that, although stent 202 is shown with the annulus portion thereof being circumferentially discontinuous in FIG. 2B, in reality the stent 202 may not achieve the same size differential shown between FIGS. 2A and 2B. This is because other structures, such as prosthetic leaflets PL and/or a cuff/skirt coupled to the stent 202, may restrict the amount that the stent 202 is actually capable of “opening” upon expansion of a balloon therein. However, the circumferential continuity of a stent may be an important limiting factor in the ability to effectively expand a secondary VIV transcatheter prosthetic heart valve into a prior-implanted prosthetic heart valve, and the ability to transition to the “open” or circumferentially discontinuous configuration shown in FIG. 2B may remove this limiting factor.

Although prosthetic heart valve 200 is described as a self-expanding valve, it should be understood that prosthetic heart valve 200 may instead be a balloon-expandable valve. If prosthetic heart valve 200 is provided as a balloon-expandable valve, it should be understood that, upon deployment, a balloon should not apply a force larger than the threshold force required to transition the stent 202 from the circumferentially continuous condition of FIG. 2A to the circumferentially discontinuous condition of FIG. 2B. Further, the secondary VIV transcatheter prosthetic heart valve may be either a self-expandable or balloon-expandable valve. If the secondary VIV transcatheter prosthetic heart valve is a self-expandable valve, a balloon dilatation or similar procedure would still be performed on the prosthetic heart valve 200 just prior to allowing the secondary VIV transcatheter prosthetic heart valve to self-expand into the prosthetic heart valve 200. If the secondary VIV transcatheter prosthetic heart valve is a balloon-expandable valve, a balloon dilatation or similar procedure may be performed on the prosthetic heart valve 200 just prior to balloon expansion of the secondary VIV transcatheter prosthetic heart valve into the prosthetic heart valve 200. However, in other embodiments, the balloon expansion of the secondary VIV transcatheter prosthetic heart valve may provide enough force to transition the stent 202 from the circumferentially continuous condition to the circumferentially discontinuous condition, so that a separate step of balloon dilatation of the stent 202 is not required. Still further, although prosthetic heart valve 200 is described above as a transcatheter prosthetic heart valve, in some embodiments the structure of connectors 203 (or similar variations thereof) may be applied to a stent of a surgical heart valve that is sutured into the native annulus in an open-chest, open-heart procedure. Those connectors 203, in a surgical prosthetic heart valve, would be similarly capable of “breaking open” upon application of a threshold force during a secondary VIV transcatheter prosthetic heart valve implantation procedure.

Finally, although prosthetic heart valve 200 is shown as a prosthetic atrioventricular valve, similar connectors 203 may be used on a prosthetic heart valve that is for replacing a native aortic or pulmonary valve. For example, the connectors 203 may be used on a generally cylindrical stent of a balloon-expandable prosthetic aortic valve, or on a self-expanding prosthetic aortic valve in which the stent has a shape generally similar to stent 102 of FIG. 1. If connectors similar to connectors 203 are used on a stent similar to stent 102, those connectors may be positioned only in the annulus section (e.g. the bottom two rows of cells) without any such connectors in row(s) of cells in the transition section or the row(s) of cells in the aortic section. However, such connectors 203 could be provided in one or both of the rows of cells in the transition section (e.g. where CAFs 116 are located) or the aortic section (e.g. the top-most row of cells in the view of FIG. 1).

According to one aspect of the disclosure, a prosthetic heart valve comprises:
a stent body extending from an inflow end to an outflow end in a longitudinal direction, the stent body including a generally tubular annulus section defining a first row of cells extending in a circumferential direction, the stent body being expandable from a delivery condition having a first diameter to a deployed condition having a second diameter larger than the first diameter; and
a plurality of prosthetic leaflets mounted to the stent body and operative to allow flow in an antegrade direction from the inflow end to the outflow end, but to substantially block flow in a retrograde direction from the outflow end to the inflow end,
wherein the first row of cells is circumferentially continuous in the delivery condition and in the deployed condition, and the stent body is further expandable from the deployed condition to an open condition in which the first row of cells is circumferentially discontinuous; and/or
a first cell in the first row of cells is coupled to a circumferentially adjacent second cell in the first row of cells by a connector, the connector configured to decouple the first cell from the second cell when a predetermined force is applied to an inner surface of the stent body while transitioning the stent body from the deployed condition to the open condition; and/or
the connector is formed by a first connector portion of the first cell, and a second connector portion of the second cell; and/or
an isolator positioned at a point of engagement between the first connector portion and the second connector portion when the stent body is in the deployed condition, the isolator configured to limit direct contact between the first connector portion and the second connector portion when the first cell is coupled to the second cell by the connector; and/or
the first connector portion and the second connector portion are each formed of metal, and the isolator is formed of tissue or fabric; and/or
the first connector portion is hook-shaped and wraps around the second connector portion when the first cell is coupled to the second cell by the connector; and/or
when the first cell is coupled to the second cell by the connector, the first connector portion includes a thinned neck portion positioned where the first connector portion wraps around the second connector portion, the thinned neck portion having a thickness that is smaller than a thickness of the first connector portion adjacent the thinned neck portion; and/or
upon application of the predetermined force to the inner surface of the stent body, the first connector portion is configured to preferentially deform at the thinned neck portion to decouple the first cell from the second cell; and/or
the second connector portion is hook-shaped, the isolator being coupled to a face of the second connector portion that confronts the first connector portion when the first cell is coupled to the second cell by the connector; and/or
the stent body is formed of a shape-memory material and is configured to self-expand from the delivery condition to the deployed condition; and/or
a first cell in a second row of cells is coupled to a circumferentially adjacent second cell in the second row of cells by a connector, the connector configured to decouple the first cell from the second cell when a predetermined force is applied to an inner surface of the stent body while transitioning the stent body from the deployed condition to the open condition; and/or
a first cell in a row of cells is coupled to a circumferentially nonadjacent second cell in a circumferentially nonadjacent row of cells by a connector, the connector configured to decouple the first cell from the second cell when a predetermined force is applied to an inner surface of the stent body while transitioning the stent body from the deployed condition to the open condition; and/or
the cells of the stent body are substantially diamond shaped with two ends pointing in the longitudinal direction and two ends pointing in the circumferential directions when in the deployed condition; and/or
the cells of the stent body are substantially diamond shaped with two ends pointing in the longitudinal direction, two ends pointing in the circumferential directions, and a connector coupling the circumferential point of the first cell to the circumferential point of the second cell.

According to another aspect of the disclosure, a method of implanting a first prosthetic heart valve comprises:
loading the first prosthetic heart valve into a sheath of a delivery device so that a stent body of the first prosthetic heart valve is maintained in a delivery condition with a first diameter;
advancing the first prosthetic heart valve through a patient until the first prosthetic heart valve is positioned adjacent a native heart valve of the patient;
deploying the first prosthetic heart valve from the sheath of the delivery device into the native heart valve of the patient so that the stent body of the first prosthetic heart valve expands to a deployed condition with a second diameter larger than the first diameter, the stent body being further expandable, upon the application of a predetermined force to an inner surface of the stent body, from the deployed condition to an open condition in which a first row of cells of the stent body is circumferentially discontinuous; and
withdrawing the delivery device from the patient and completing the implantation of the first prosthetic heart valve without further expanding the stent body from the deployed condition to the open condition; and/or
inserting a secondary device into the patient, positioning the secondary device within the first prosthetic heart valve, and using the secondary device to apply the predetermined force to the inner surface of the stent body to transition the stent body from the deployed condition to the open condition; and/or
inserting the secondary device into the patient is performed as part of an implantation of a second prosthetic heart valve being performed after completing the implantation of the first prosthetic heart valve; and/or
a connector couples a first cell in the first row of cells to a circumferentially adjacent second cell in the first row of cells, and transitioning the stent body from the deployed condition to the open condition includes disengaging the connector to decouple the first cell from the second cell; and/or
the secondary device is a balloon catheter.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
a stent body extending from an inflow end to an outflow end in a longitudinal direction, the stent body including a generally tubular annulus section defining a first row of cells extending in a circumferential direction, the stent body being expandable from a delivery condition having a first diameter to a deployed condition having a second diameter larger than the first diameter; and
a plurality of prosthetic leaflets mounted to the stent body and operative to allow flow in an antegrade direction from the inflow end to the outflow end, but to substantially block flow in a retrograde direction from the outflow end to the inflow end,
wherein the first row of cells is circumferentially continuous in the delivery condition and in the deployed condition, and the stent body is further expandable from the deployed condition to an open condition in which the first row of cells is circumferentially discontinuous.

2. The prosthetic heart valve of claim 1, wherein a first cell in the first row of cells is coupled to a circumferentially adjacent second cell in the first row of cells by a connector, the connector configured to decouple the first cell from the second cell when a predetermined force is applied to an inner surface of the stent body while transitioning the stent body from the deployed condition to the open condition.

3. The prosthetic heart valve of claim 2, wherein the connector is formed by a first connector portion of the first cell, and a second connector portion of the second cell.

4. The prosthetic heart valve of claim 3, further comprising an isolator positioned at a point of engagement between the first connector portion and the second connector portion when the stent body is in the deployed condition, the isolator configured to limit direct contact between the first connector portion and the second connector portion when the first cell is coupled to the second cell by the connector.

5. The prosthetic heart valve of claim 4, wherein the first connector portion and the second connector portion are each formed of metal, and the isolator is formed of tissue or fabric.

6. The prosthetic heart valve of claim 4, wherein the first connector portion is hook-shaped and wraps around the second connector portion when the first cell is coupled to the second cell by the connector.

7. The prosthetic heart valve of claim 6, wherein when the first cell is coupled to the second cell by the connector, the first connector portion includes a thinned neck portion positioned where the first connector portion wraps around the second connector portion, the thinned neck portion having a thickness that is smaller than a thickness of the first connector portion adjacent the thinned neck portion.

8. The prosthetic heart valve of claim 7, wherein upon application of the predetermined force to the inner surface of the stent body, the first connector portion is configured to preferentially deform at the thinned neck portion to decouple the first cell from the second cell.

9. The prosthetic heart valve of claim 8, wherein the second connector portion is hook-shaped, the isolator being coupled to a face of the second connector portion that confronts the first connector portion when the first cell is coupled to the second cell by the connector.

10. The prosthetic heart valve of claim 1, wherein the stent body is formed of a shape-memory material and is configured to self-expand from the delivery condition to the deployed condition.

11. The prosthetic heart valve of claim 2, wherein a first cell in a second row of cells is coupled to a circumferentially adjacent second cell in the second row of cells by a connector, the connector configured to decouple the first cell from the second cell when a predetermined force is applied to an inner surface of the stent body while transitioning the stent body from the deployed condition to the open condition.

12. The prosthetic heart valve of claim 1, wherein a first cell in a row of cells is coupled to a circumferentially nonadjacent second cell in a circumferentially nonadjacent row of cells by a connector, the connector configured to decouple the first cell from the second cell when a predetermined force is applied to an inner surface of the stent body while transitioning the stent body from the deployed condition to the open condition.

13. The prosthetic heart valve of claim 1, wherein the cells of the stent body are substantially diamond shaped with two ends pointing in the longitudinal direction and two ends pointing in the circumferential directions when in the deployed condition.

14. The prosthetic heart valve of claim 2, wherein the cells of the stent body are substantially diamond shaped with two ends pointing in the longitudinal direction, two ends pointing in the circumferential directions, and a connector coupling the circumferential point of the first cell to the circumferential point of the second cell.

15. A method of implanting a first prosthetic heart valve, the method comprising:
loading the first prosthetic heart valve into a sheath of a delivery device so that a stent body of the first prosthetic heart valve is maintained in a delivery condition with a first diameter;
advancing the first prosthetic heart valve through a patient until the first prosthetic heart valve is positioned adjacent a native heart valve of the patient;
deploying the first prosthetic heart valve from the sheath of the delivery device into the native heart valve of the patient so that the stent body of the first prosthetic heart valve expands to a deployed condition with a second diameter larger than the first diameter, the stent body being further expandable, upon the application of a predetermined force to an inner surface of the stent body, from the deployed condition to an open condition in which a first row of cells of the stent body is circumferentially discontinuous; and
withdrawing the delivery device from the patient and completing the implantation of the first prosthetic heart valve without further expanding the stent body from the deployed condition to the open condition.

16. The method of claim 15, further comprising inserting a secondary device into the patient, positioning the secondary device within the first prosthetic heart valve, and using the secondary device to apply the predetermined force to the inner surface of the stent body to transition the stent body from the deployed condition to the open condition.

17. The method of claim 16, wherein inserting the secondary device into the patient is performed as part of an implantation of a second prosthetic heart valve being performed after completing the implantation of the first prosthetic heart valve.

18. The method of claim 17, wherein a connector couples a first cell in the first row of cells to a circumferentially adjacent second cell in the first row of cells, and transitioning the stent body from the deployed condition to the open condition includes disengaging the connector to decouple the first cell from the second cell.

19. The method of claim 16, wherein the secondary device is a balloon catheter.
